(19) United States
(12) Patent Application Publication
(10) Pub. No.: US 2024/0312634 A1
(43) Pub. Date: Sep. 19, 2024

(54) RISK RELATIONSHIP MENTAL HEALTH EQUIPMENT MANAGEMENT SYSTEM

(71) Applicants: Wei Wang, South Glastonbury, CT (US); Moira E. Pierce, Southington, CT (US); Matthew W. Brown, Cromwell, CT (US)

(72) Inventors: Wei Wang, South Glastonbury, CT (US); Moira E. Pierce, Southington, CT (US); Matthew W. Brown, Cromwell, CT (US)

(21) Appl. No.: 18/183,493

(22) Filed: Mar. 14, 2023

(51) Int. Cl. G16H 50/30; G16H 10/60

(52) U.S. Cl. CPC G16H 50/30; G16H 10/60

(57) ABSTRACT

A mental health equipment data store may contain electronic records associated with mental health equipment identifiers. For each mental health equipment identifier, the data store may include a communication address and associated equipment parameters. A risk relationship data store may contain electronic records associated with parties having risk relationships with an enterprise. A back-end application computer server may associate a selected equipment identifier in the mental health equipment data store with a selected party having a risk relationship with the enterprise. The server may update the risk relationship data store with the selected equipment identifier and arrange to collect selected mental health data for the selected party in accordance with the communication address of the selected equipment identifier. The selected mental health data is input to a predictive model algorithm, and the server automatically executes a treatment workflow for the selected party based on an output of the algorithm.

### TECHNICAL FIELD

The present application generally relates to computer systems and more particularly to computer systems that are adapted to accurately, securely, and/or automatically manage mental health equipment for a risk relationship enterprise.

### BACKGROUND

An enterprise may enter into relationships with various entities and parties. For example, an insurer might enter into risk relationships (e.g., insurance agreements) with various businesses and/or employees. In some cases, enterprise may arrange to provide preventive benefits and/or treatments (e.g., to reduce workplace injuries and/or help an employee with a disability return to work). Moreover, employee mental health is one of the top employer priorities in the workplace. According to studies, mental disorders in the workforce are rising (e.g., in 2022 nearly 20% of adults in the US experienced a mental illness—nearly 50 million Americans). In addition, 25% have left a job due to mental health concerns and over 10% of functional disabilities may be caused by mental health issues. Although various equipment is available to help monitor and treat mental health, employers and employees may be unaware of (or unable to access) such equipment. It can be difficult for an insurer to help employees learn about and use these devices.

It would be desirable to provide improved systems and methods to accurately and/or automatically provide mental health equipment management and management tools for an enterprise. Moreover, the results should be easy to access, understand, interpret, update, etc.

### SUMMARY OF THE INVENTION

According to some embodiments, systems, methods, apparatus, computer program code and means are provided to accurately and/or automatically provide mental health equipment management and management tools for an enterprise in a way that provides fast, secure, and useful results and that allows for flexibility and effectiveness when responding to those results.

Some embodiments are directed to a mental health equipment management and/or management tool implemented via a back-end application computer server. A mental health equipment data store may contain electronic records associated with mental health equipment identifiers. For each mental health equipment identifier, the data store may include a communication address and associated equipment parameters. A risk relationship data store may contain electronic records associated with parties having risk relationships with an enterprise. A back-end application computer server may associate a selected equipment identifier in the mental health equipment data store with a selected party having a risk relationship with the enterprise. The server may update the risk relationship data store with the selected equipment identifier and arrange to collect selected mental health data for the selected party in accordance with the communication address of the selected equipment identifier. The selected mental health data is input to a predictive model algorithm, and the server automatically executes a treatment workflow for the selected party based on an output of the algorithm.

Some embodiments comprise: means for associating, by a computer processor of a back-end application computer server, a selected equipment identifier in a mental health equipment data store with a selected party having a risk relationship with the enterprise, the mental health equipment data store containing electronic records associated with a plurality of mental health equipment identifiers, and, for each mental health equipment identifier, a communication address and associated equipment parameters; means for updating a risk relationship data store with the selected equipment identifier, the risk relationship data store containing electronic records associated with a plurality of parties having risk relationships with the enterprise, and, for each party, a party identifier, at least one mental health equipment identifier, and mental health data; means for arranging to collect selected mental health data for the selected party in accordance with the communication address of the selected equipment identifier; means for inputting the selected mental health data to a predictive model algorithm; and means for automatically executing a treatment workflow for the selected party based on an output of the predictive model algorithm.

In some embodiments, a communication device associated with a back-end application computer server exchanges information with remote devices in connection with interactive graphical user interfaces. The information may be exchanged, for example, via public and/or proprietary communication networks.

A technical effect of some embodiments of the invention is improved and computerized mental health equipment management and/or management tools for an enterprise that provide fast, secure, and useful results. With these and other advantages and features that will become hereinafter apparent, a more complete understanding of the nature of the invention can be obtained by referring to the following detailed description and to the drawings appended hereto.

### BRIEF DESCRIPTION OF THE DRAWINGS

FIG. 1 shows various enterprise risk relationships according to some embodiments.

FIG. 2 is a high-level block diagram of a mental health equipment management and/or management system in accordance with some embodiments.

FIG. 3 illustrates a high-level method according to some embodiments.

FIG. 4 is a system overview in accordance with some embodiments.

FIG. 5 is an employee mental health support framework according to some embodiments.

FIG. 6 is an example of a mental health smartphone application display in accordance with some embodiments.

FIG. 7 is an equipment management system smartphone display according to some embodiments.

FIG. 8 is an equipment management system web display in accordance with some embodiments.

FIG. 9 is a more detailed system according to some embodiments.

FIG. 10 is a multi-tier implementation in accordance with some embodiments.

FIG. 11 is a wearable mental health device according to some embodiments.

FIG. 12 is a treatment workflow method in accordance with some embodiments.

FIG. 13 is a block diagram of an apparatus in accordance with some embodiments.

FIG. 14 is a portion of a tabular mental health equipment database according to some embodiments.

FIG. 15 is a portion of a mental health database according to some embodiments.

FIG. 16 is a portion of a tabular insurance policy database according to some embodiments.

FIG. 17 is an operator or administrator display in accordance with some embodiments.

FIG. 18 illustrates a tablet computer with an equipment management tool display according to some embodiments.

FIG. 19 illustrates a system having a predictive model in accordance with some embodiments.

### DETAILED DESCRIPTION

Before the various exemplary embodiments are described in further detail, it is to be understood that the present invention is not limited to the particular embodiments described. It is also to be understood that the terminology used herein is for the purpose of describing particular embodiments only and is not intended to limit the scope of the claims of the present invention.

In the drawings, like reference numerals refer to like features of the systems and methods of the present invention. Accordingly, although certain descriptions may refer only to certain figures and reference numerals, it should be understood that such descriptions might be equally applicable to like reference numerals in other figures.

The present invention provides significant technical improvements to facilitate data processing associated with mental health equipment management. The present invention is directed to more than merely a computer implementation of a routine or conventional activity previously known in the industry as it provides a specific advancement in the area of electronic record analysis by providing improvements in the operation of a computer system that customizes mental health equipment management (including those associated with risk relationships). The present invention provides improvement beyond a mere generic computer implementation as it involves the novel ordered combination of system elements and processes to provide improvements in the speed, security, and accuracy of such an equipment management tool for an enterprise. Some embodiments of the present invention are directed to a system adapted to automatically customize and execute equipment management, aggregate data from multiple data sources, automatically optimize equipment information to reduce unnecessary messages or communications, etc. (e.g., to consolidate mental health data). Moreover, communication links and messages may be automatically established, aggregated, formatted, modified, removed, exchanged, etc. to improve network performance (e.g., by reducing an amount of network messaging bandwidth and/or storage required to create equipment management messages or alerts, improve security, reduce the size of a mental health equipment data store, more efficiently collect mental health data, etc.).

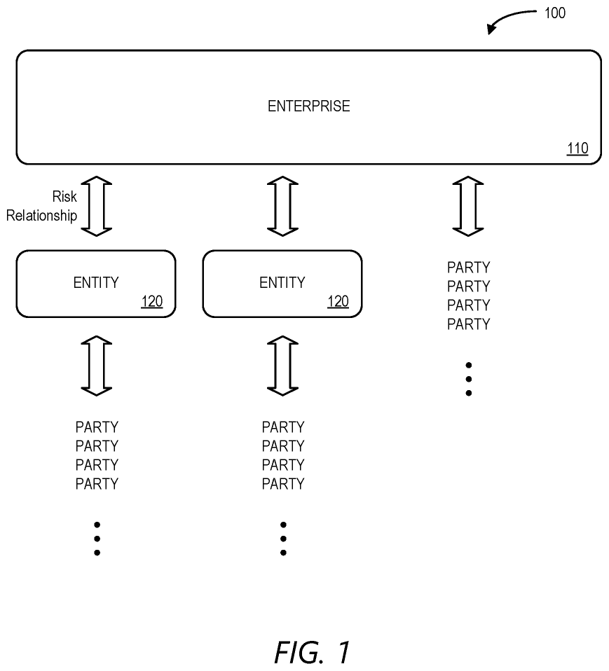

FIG. 1 shows various enterprise risk relationships 100 according to some embodiments. In particular, an enterprise 110, such as an insurance company, may have risk relationships (e.g., insurance agreements or policies) associated with parties (either directly with the parties or through entities 120 such as employers). For example, an insurance company may help an employer offer Employee Assistance Program (“EAP”) benefits for employees. As used herein, the term EAP might refer to work-based intervention programs help employees detect and/or resolve problems, including mental health issues, that may be adversely affecting performance. An EAP may also include a wide array services, such as nurse advice lines, that might be made available not only to the employee but also to the employee's spouse, children, non-marital partner living in the same household as the employee, etc.

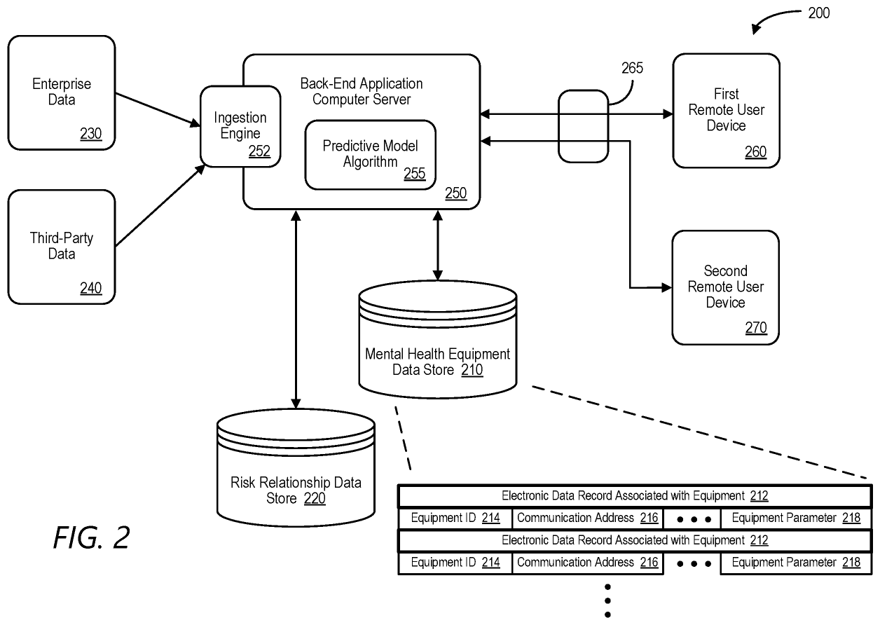

FIG. 2 is a high-level block diagram of a mental health equipment management and/or management system 200 that may be provided according to some embodiments of the present invention. In particular, the system 200 includes a back-end application computer server 250 that may access information in a mental health equipment data store 210 (e.g., storing a set of electronic records associated with various types of equipment 212, each record including, for example, one or more equipment identifiers 214, communication addresses 216, equipment parameters 218, etc.). The back-end application computer server 250 may also store information into other data stores, such as a risk relationship data store 220, and utilize an ingestion engine 252 and predictive model algorithm 255 to exchange and process messages (e.g., daily/weekly data sweeps or on-demand changes) and view, analyze, and/or update the electronic records. The back-end application computer server 250 may also exchange information with a first remote user device 260 and a second remote user device 270 (e.g., via a firewall 265). According to some embodiments, an interactive graphical user interface platform of the back-end application computer server 250 (and, in some cases, enterprise data 230 and/or third-party data 240) may facilitate equipment management, treatment workflow recommendations, alerts, and/or the display of results via one or more remote administrator computers (e.g., to summarize system 200 performance) and/or the remote user devices 260, 270. For example, the first remote user device 260 may transmit annotated and/or updated information to the back-end application computer server 250. Based on the updated information, the back-end application computer server 250 may adjust data in the mental health equipment data store 210 and/or the risk relationship data store 220 and the change may (or may not) be used in connection with the second remote user device 270 (e.g., depending on whether the two users are associated with the same enterprise or entity). Note that the back-end application computer server 250 and/or any of the other devices and methods described herein might be associated with a third party, such as a vendor that performs a service for an enterprise.

The back-end application computer server 250 and/or the other elements of the system 200 might be, for example, associated with a Personal Computer (“PC”), laptop computer, smartphone, an enterprise server, a server farm, and/or a database or similar storage devices. According to some embodiments, an “automated” back-end application computer server 250 (and/or other elements of the system 200) may facilitate the automated access and/or update of electronic records in the data stores 210, 220 and/or the management of mental health equipment. As used herein, the term “automated” may refer to, for example, actions that can be performed with little (or no) intervention by a human.

Devices, including those associated with the back-end application computer server 250 and any other device described herein, may exchange information via any communication network which may be one or more of a Local Area Network (“LAN”), a Metropolitan Area Network (“MAN”), a Wide Area Network (“WAN”), a proprietary network, a Public Switched Telephone Network (“PSTN”), a Wireless Application Protocol (“WAP”) network, a Bluetooth network, a wireless LAN network, and/or an Internet Protocol (“IP”) network such as the Internet, an intranet, or an extranet. Note that any devices described herein may communicate via one or more such communication networks.

The back-end application computer server 250 may store information into and/or retrieve information from the mental health equipment data store 210 and/or the risk relationship data store 220. The data stores 210, 220 may be locally stored or reside remote from the back-end application computer server 250. As will be described further below, the mental health equipment data store 210 may be used by the back-end application computer server 250 in connection with an interactive user interface to access and update electronic records. Although a single back-end application computer server 250 is shown in FIG. 2, any number of such devices may be included. Moreover, various devices described herein might be combined according to embodiments of the present invention. For example, in some embodiments, the back-end application computer server 250 and mental health equipment data store 210 might be co-located and/or may comprise a single apparatus.

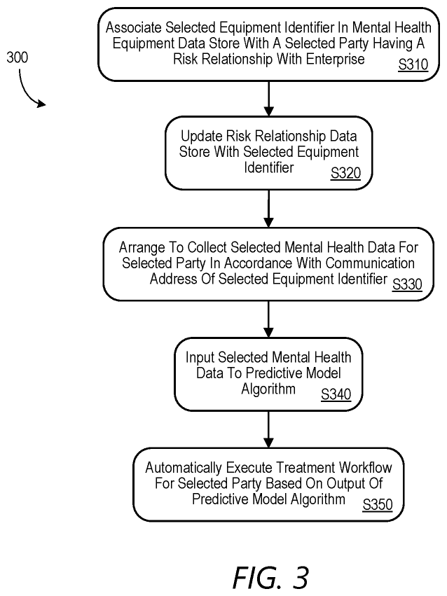

The elements of the system 200 may work together to perform the various embodiments of the present invention. Note that the system 200 of FIG. 2 is provided only as an example, and embodiments may be associated with additional elements or components. According to some embodiments, the elements of the system 200 automatically transmit information associated with an interactive user interface display over a distributed communication network. FIG. 3 illustrates a method 300 that might be performed by some or all of the elements of the system 200 described with respect to FIG. 2, or any other system, according to some embodiments of the present invention. The flow charts described herein do not imply a fixed order to the steps, and embodiments of the present invention may be practiced in any order that is practicable. Note that any of the methods described herein may be performed by hardware, software, or any combination of these approaches. For example, a computer-readable storage medium may store thereon instructions that when executed by a machine result in performance according to any of the embodiments described herein.

At S310, a back-end application computer server may associate a selected equipment identifier in a mental health equipment data store with a selected party having a risk relationship with the enterprise. The mental health equipment data store may, for example, contain electronic records associated with a plurality of mental health equipment identifiers. For each mental health equipment identifier, the mental health equipment data store may include a communication address and associated equipment parameters (e.g., parameters describe and/or define operation of the equipment).

The selected equipment identifier might be based on, according to some embodiments, a request from a party. For example, an employee might indicate that he or she has purchased, installed, and/or is using the device. In other cases, the selected equipment identifier might be based on a request from an entity associated with the party and an enterprise (e.g., an employer or business) or an automatic analysis of mental health data (e.g., a predictive model algorithm might automatically recommend a particular device for a particular employee).

Note that mental health equipment might refer to various types of devices, including a smartphone, a smartwatch, augmented reality glasses, a virtual reality display, an interactive application, a web site, etc. According to some embodiments, at least one equipment identifier is associated with wearable equipment such as a smartwatch, a bracelet, a ring, a vest, a headband, a pillow, a mattress, etc. Note that these are provided only as examples and embodiments may be associated with any other type of apparatus capable of sensing and/or transmitting data that might be associated with mental health, including safety devices such as hats, goggles, or helmets, gloves, jewelry, an identification credential (e.g., a badge or lanyard), a dongle attached to a keychain or USB port (e.g., to track location and/or movement), etc.

At S320, a risk relationship data store may be updated with the selected equipment identifier. The risk relationship data store may, for example, contain electronic records for a plurality of parties that have risk relationships with the enterprise. For each party, the risk relationship data store may include a party identifier, at least one mental health equipment identifier, and mental health data (e.g., data sensed by the mental health equipment).

At S330, the server may arrange to collect selected mental health data for the selected party in accordance with the communication address of the selected equipment identifier. As used herein, the term communication address may refer to any information that lets an enterprise exchange information with mental health equipment, including a telephone number, an email address, an IP address, Internet of Things (“IoT”) information, etc. At S340, the selected mental health data is input to a predictive model algorithm, and the system automatically executes a treatment workflow for the selected party based on an output of the predictive model algorithm at S350. The treatment workflow might be, for example, associated with an application (a smartphone application with a chat interface), a return-to-work strategy, a treatment referral, an EAP, etc. According to some embodiments, the enterprise comprises an insurance company and each party comprises an employee of a business. In this case, the risk relationship might be associated with, for example, an insurance policy (e.g., a group benefit), workers' compensation, Long Term Disability (“LTD”). Short Term Disability (“STD”), etc.

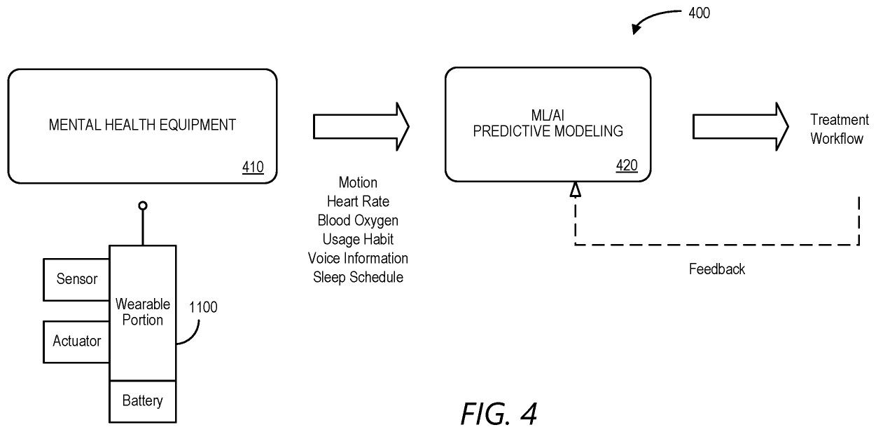

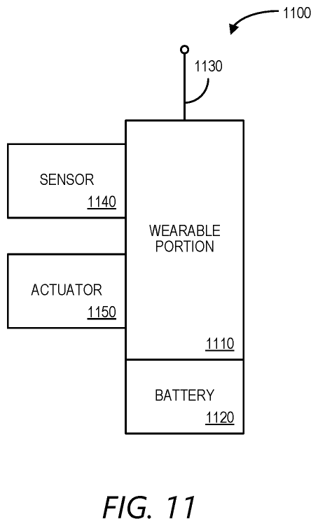

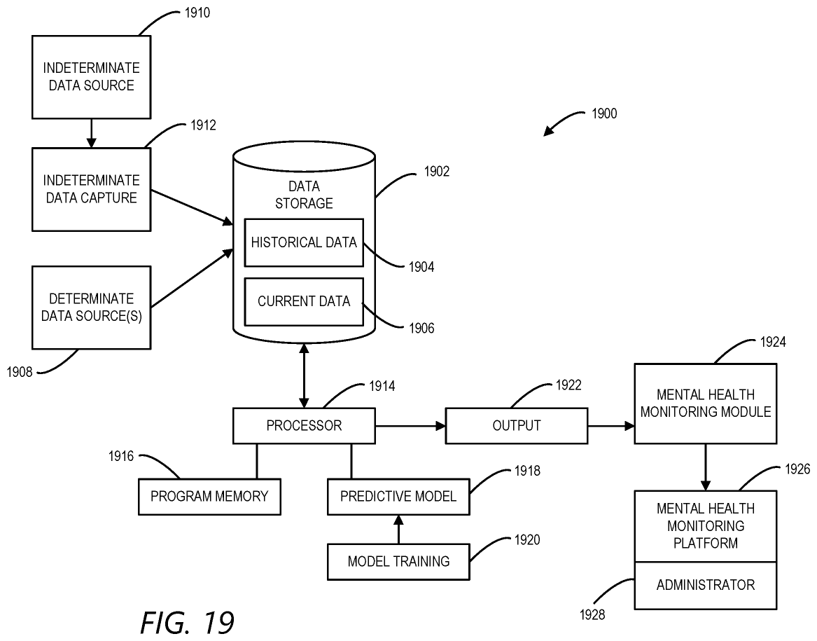

FIG. 4 is a system overview 400 in accordance with some embodiments. Mental health equipment 410 may provide mental health data to a Machine Learning (“ML”)/Artificial Intelligence (“AI”) predictive modeling 420 (described in detail with respect to FIG. 19). The mental health equipment 410 may, in some embodiments, comprise a wearable device 1100 (described in detail with respect to FIG. 11). The mental health data might include, for example, information about motion, heart rate, blood oxygen, usage habits (e.g., an amount of smartphone usage), voice information, a sleep schedule, etc. The predictive modeling 420 may recommend a treatment workflow (e.g., automatically sending an email about an EAP to an employee). The result of the predictive modeling 420 and/or the treatment workflow may be provided to the predictive modeling 420 as feedback to improve performance over time. The treatment workflow might range from meditation programs (e.g., for employees who want to improve their ability to focus, need to relax, etc.) to an EAP referral (e.g., for depression). In between those extremes, treatment workflows might help employees stress and anxiety levels, improve sleep patterns, increase confidence and self-esteem, address pain and relationship issues, etc.).

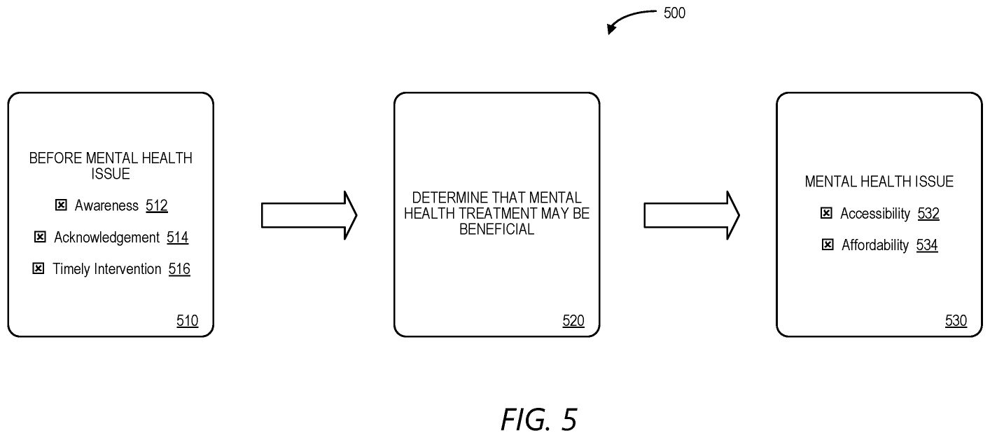

FIG. 5 is an employee mental health support framework 500 according to some embodiments. During a period before a mental health issue arises 510, the framework 500 may encourage awareness 512, acknowledgement 514 (e.g., making sure an employee is aware they are experiencing mental health warning signs), and/or timely intervention 516 regarding mental health. After it is determined that mental health treatment may be beneficial 520 support may be provided for the mental health issue 530 with a focus on accessibility 532 and affordability 534.

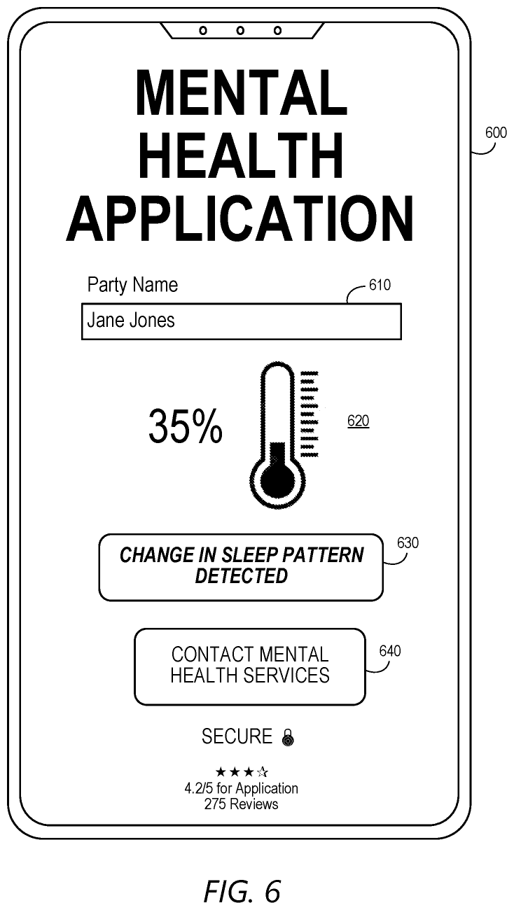

In some cases, the mental health equipment might comprise a smartphone application. For example, FIG. 6 is an example of a mental health smartphone application display 600 in accordance with some embodiments. The display 600 includes a party name or identifier 610 and an overall mental health score 620. The display 600 might further include alerts 630 and/or provide a way for an employee to automatically and securely connect with mental health services 640. According to some embodiments, a smartphone application might include an AI chat interface to help an employee feel heard and/or understood.

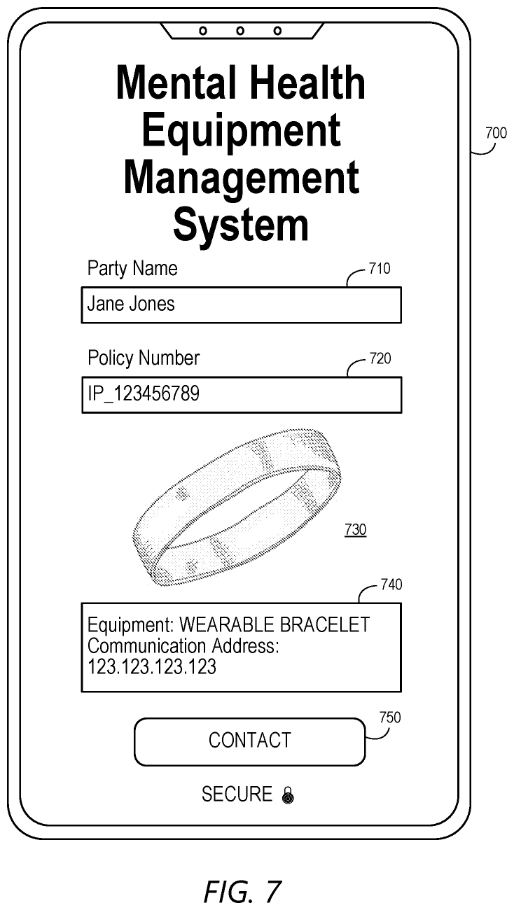

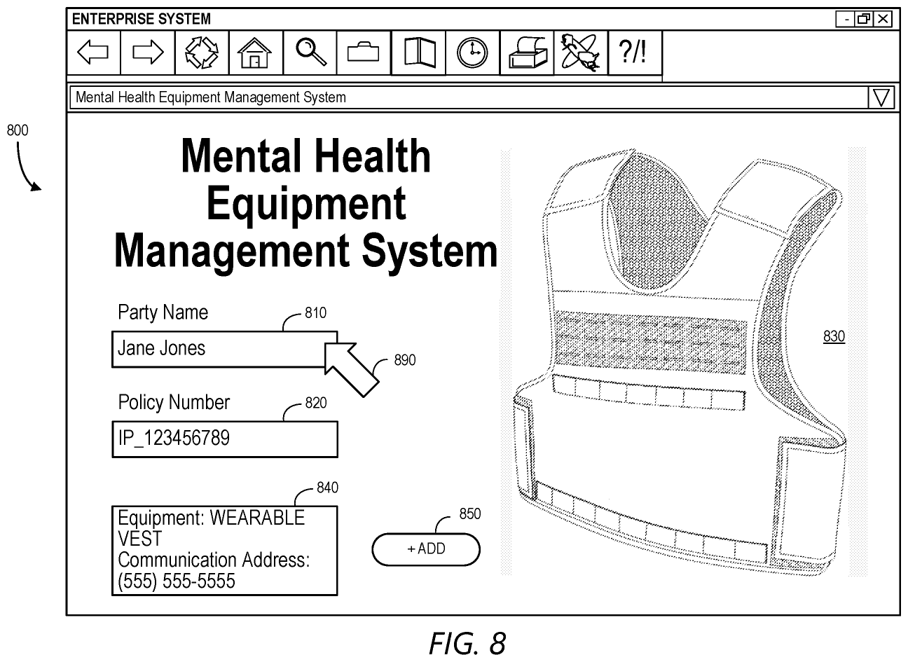

FIG. 7 is an equipment management system smartphone display 700 according to some embodiments. The display 700 might let an employee (e.g., a party 710 associated with an insurance policy 720) add mental health equipment 730 to his or her inventory of equipment. The equipment 730 may include a description 740 of the device including a communication address and a way for an employee, employer, or insurer to easily establish contact 750 as appropriate (e.g., to report or suggest a particular device). As another example, FIG. 8 is an equipment management system web display 800 in accordance with some embodiments. As before, the display 800 might let an employee (e.g., a party 810 associated with an insurance policy 820) view mental health equipment 830 including a description 840 of the device with a communication address. The party 810 may select the equipment 830 to be imported into his or her inventory (e.g., with an “+Add” icon 850 via a touchscreen or computer mouse pointer 890).

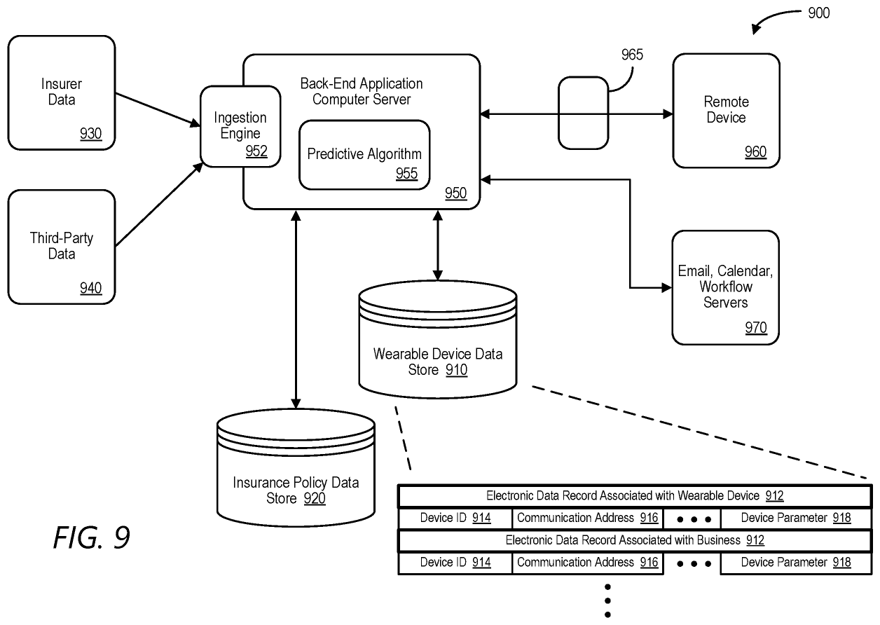

FIG. 9 is a more detailed system 900 according to some embodiments. As before, the system 900 includes a back-end application computer server 950 that may access information in a wearable device data store 910 (e.g., storing a set of electronic records associated with wearable device 912, each record including, for example, one or more device identifiers 914, communication addresses 916, device parameters 918, etc.). The back-end application computer server 950 may also store information into other data stores, such as insurance policy data store 920, and utilize an ingestion engine 952 and predictive algorithm 955 to exchange and process mental health changes (e.g., daily/weekly data sweeps or on-demand changes) and view, analyze, and/or update the electronic records based on insurer data 930, third-party data 940, etc. The back-end application computer server 950 may also exchange information with a remote device 960 (e.g., via a firewall 965). According to some embodiments, the back-end application computer server 950 may interact with an email server (e.g., to automatically establish communication links and/or transmit electronic messages), a calendar server (e.g., to automatically schedule tasks or communications), and/or a workflow server 970 (e.g., to initiate actions by employees or programs of the enterprise).

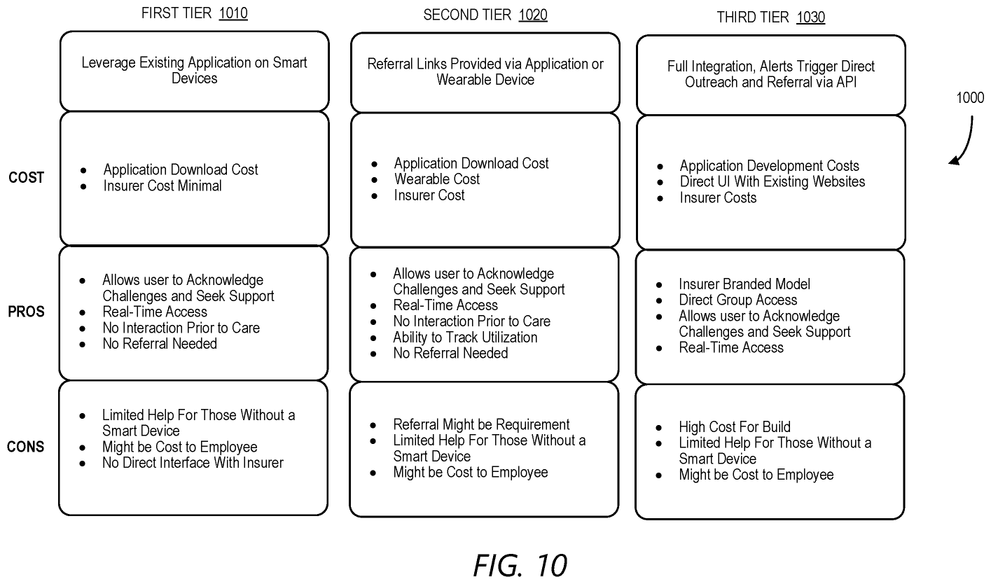

The information collected and processed by the system 900 may then be used to facilitate mental health programs established in connection with insurance policies and/or employees. For example, FIG. 10 is a multi-tier implementation 1000 in accordance with some embodiments. A first tier 1010 may be associated with a program that leverages existing third-party mental health applications on smart devices. For example, a third-party application may interact with an employee to help gauge his or her mental health condition. A second tier 1020 may provide mental health treatment referral links via a smartphone application or wearable device. A third tier 1030 may fully integrate mental health data with the enterprise and/or trigger alerts to allow for direct outreach to employees, automatic EAP referrals via an Application Programming Interface (“API”), etc.

FIG. 11 is a wearable mental health device 1100 according to some embodiments. The wearable health device 1100 includes a wearable portion 1110 (e.g., an arm band, a vest, a glove, etc.), a battery 1120 or other power source, and an antenna 1130 (e.g., to communicate with a smartphone, WiFi, the internet, etc.). A sensor 1140 may detect mental health parameters (e.g., a pulse, sleep patterns, movement, etc.) and an actuator 1150 may provide mental health treatment to the wearer (e.g., vibrations, soothing sounds, heat, etc.). In some embodiments, the sensor 1140 might comprise a microphone to help analyze voice data, a location tracker to help monitor movement, a video camera to help analyze images, etc.

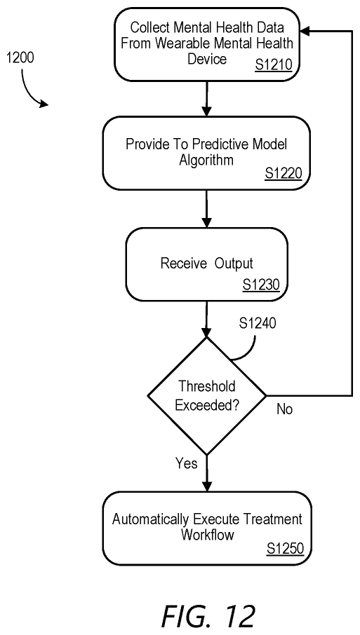

FIG. 12 is a treatment workflow method 1200 in accordance with some embodiments. At S1210, the system may collect mental health data from a wearable mental health device. For example, heart rate data, breathing rates, etc. may be transmitted to an enterprise. At S1220, the mental health data is provided to a predictive algorithm and an output is received from the algorithm at S1230 (e.g., a mental health score or array of scores). If the output does not exceed a threshold value at S1240, the system continues to monitor the data from the wearable mental health device at S1210. If the output exceeds a threshold value at S1240, an appropriate treatment workflow is automatically selected and executed at S2150. For example, an employee might receive an automated text message reminding them of mental health services that are available from an employer or insurer.

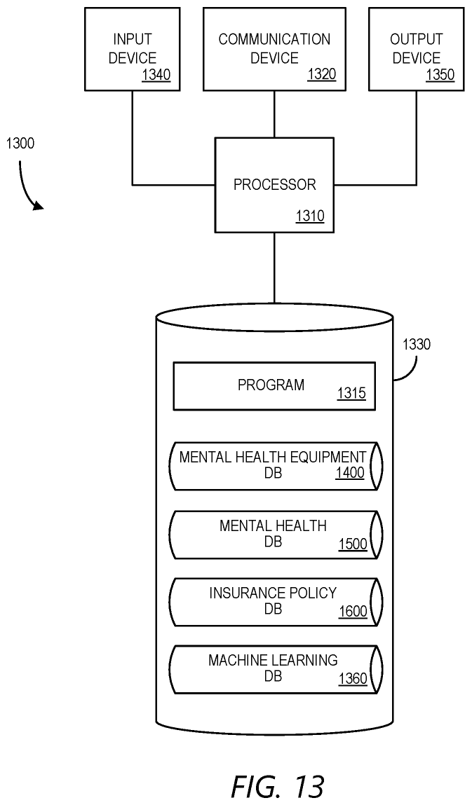

The embodiments described herein may be implemented using any number of different hardware configurations. For example, FIG. 13 illustrates an apparatus 1300 that may be, for example, associated with the systems 200, 900 described with respect to FIGS. 2 and 9, respectively. The apparatus 1300 comprises a processor 1310, such as one or more commercially available Central Processing Units (“CPUs”) in the form of one-chip microprocessors, coupled to a communication device 1320 configured to communicate via a communication network (not shown in FIG. 13). The communication device 1320 may be used to communicate, for example, with one or more remote third-party, employee, administrator, insurance agent, and/or communication devices (e.g., PCs and smartphones). Note that communications exchanged via the communication device 1320 may utilize security features, such as those between a public internet user and an internal network of an insurance company and/or an enterprise. The security features might be associated with, for example, web servers, firewalls, and/or PCI infrastructure. The apparatus 1300 further includes an input device 1340 (e.g., a mouse and/or keyboard to enter information about mental health data sources, equipment management rules or preferences, a third-party, etc.) and an output device 1350 (e.g., to output reports regarding user equipment management, machine learning algorithms, recommendations, alerts, etc.).

The processor 1310 also communicates with a storage device 1330. The storage device 1330 may comprise any appropriate information storage device, including combinations of magnetic storage devices (e.g., a hard disk drive), optical storage devices, mobile telephones, and/or semiconductor memory devices. The storage device 1330 stores a program 1315 and/or an equipment management tool or application for controlling the processor 1310. The processor 1310 performs instructions of the program 1315, and thereby operates in accordance with any of the embodiments described herein. For example, the processor 1310 may associate a selected equipment identifier with a selected party having a risk relationship with an enterprise. The processor 1310 may update risk relationship information with the selected equipment identifier and arrange to collect selected mental health data for the selected party in accordance with a communication address. The selected mental health data may be input to a predictive model algorithm, and the processor 1310 can automatically execute a treatment workflow for the selected party based on an output of the algorithm.

The program 1315 may be stored in a compressed, uncompiled and/or encrypted format. The program 1315 may furthermore include other program elements, such as an operating system, a database management system, and/or device drivers used by the processor 1310 to interface with peripheral devices.

As used herein, information may be “received” by or “transmitted” to, for example: (i) the apparatus 1300 from another device; or (ii) a software application or module within the apparatus 1300 from another software application, module, or any other source.

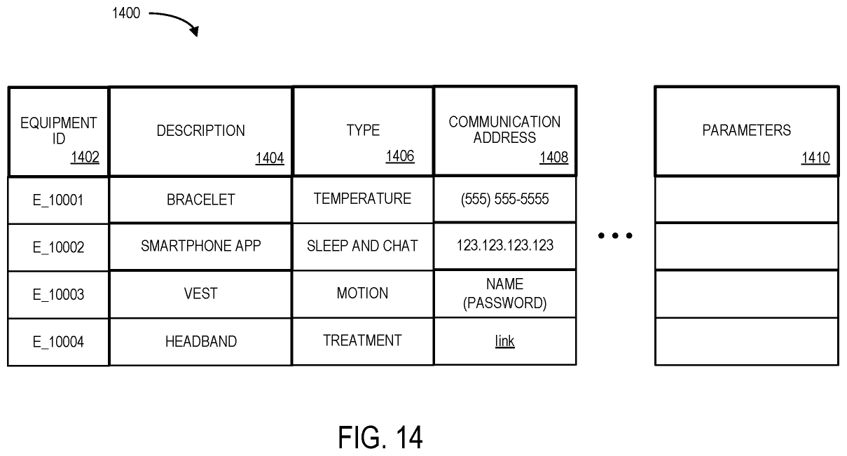

In some embodiments (such as shown in FIG. 13), the storage device 1330 further includes a mental health equipment database 1400, a mental health database 1500, an insurance policy database 1600, and a machine learning database 1360 (e.g., storing predictive models for various types of employees, equipment, etc.). Examples of databases that might be used in connection with the apparatus 1300 will now be described in detail with respect to FIGS. 14 through 16. Note that the databases described herein are only examples, and additional and/or different information may be stored therein. Moreover, various databases might be split or combined in accordance with any of the embodiments described herein. For example, the mental health equipment database 1400 and insurance policy database 1600 might be combined and/or linked to each other within the program 1315.

Referring to FIG. 14, a table is shown that represents the mental health equipment database 1400 that may be stored at the apparatus 1300 according to some embodiments. The table may include, for example, entries associated with different types of wearable device that may be used to collect mental health related information from employees. The table may also define fields 1402, 1404, 1406, 1408, 1410 for each of the entries. The fields 1402, 1404, 1406, 1408, 1410 may, according to some embodiments, specify: an equipment identifier 1402, description 1404, a type 1406, a communication address 1408, and parameters 1410. The mental health equipment database 1400 may be created and updated, for example, based on information electrically received from various equipment providers (e.g., including when a new type of device is added or existing information is adjusted) in connection with an insurer or employer.

The equipment identifier 1402 may be, for example, a unique alphanumeric code identifying a type of wearable device or other apparatus that can collect mental health information. The description 1404 may categorize the device and the type 1406 may reflect the kinds of information that is collected by the equipment. The communication address 1408 may be used, for example, to automatically connect the enterprise with the device. The parameters 1410 may indicate how information is formatted, how various health scores are defined, etc. and may be used to help collect data, select appropriate treatment workflows, etc.

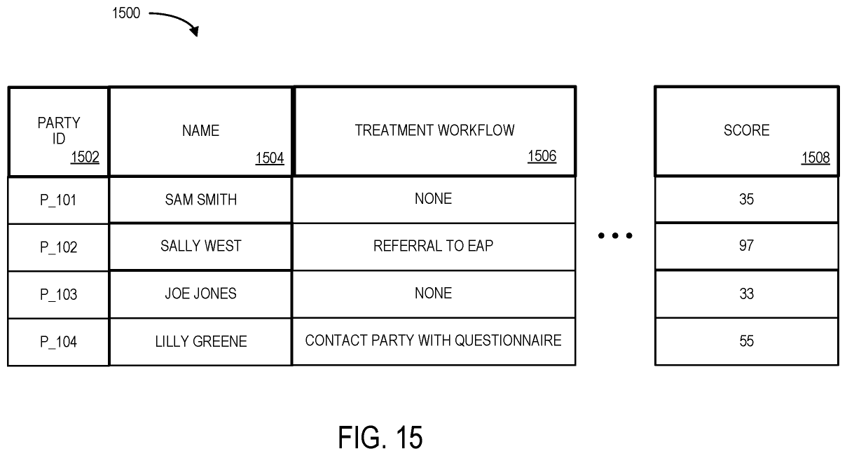

Referring to FIG. 15, a table is shown that represents the mental health database 1500 that may be stored at the apparatus 1300 according to some embodiments. The table may include, for example, entries associated mental health information collected about employees. The table may also define fields 1502, 1504, 1506, 1508 for each of the entries. The fields 1502, 1504, 1506, 1508 may, according to some embodiments, specify: a party identifier 1502, a name 1504, a treatment workflow 1506, and a score 1508. The mental health database 1500 may be created and updated, for example, based on information electrically received from mental health equipment.

The party identifier 1502 may be, for example, a unique alphanumeric code identifying an employee having a risk relationship with an enterprise. The name 1504 may be the employee's name and the treatment workflow 1506 may identify an automated process that has been selected for the employee. The score 1508 may comprise an overall mental health value calculated for the employee (e.g., calculated by a mental health device or predictive algorithm). For example, employees having an overall mental health score 1508 with a value greater than “50” might be considered “at risk” and assigned a treatment workflow 1506.

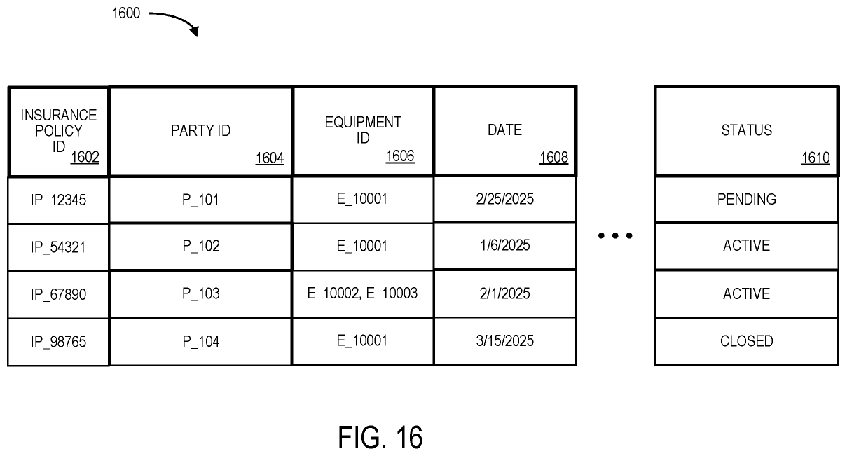

Referring to FIG. 16, a table is shown that represents the insurance policy database 1600 that may be stored at the apparatus 1300 according to some embodiments. The table may include, for example, entries associated with risk relationships between an enterprise and various entities and parties. The table may also define fields 1602, 1604, 1606, 1608, 1610 for each of the entries. The fields 1602, 1604, 1606, 1608, 1610 may, according to some embodiments, specify: an insurance policy identifier 1602, a party identifier 1604, an equipment identifier 1606, a date 1608, and a status 1610. The insurance policy database 1600 may be created and updated, for example, based on local enterprise data and/or information that has been electrically received from various remote devices in connection with an insurer.

The insurance policy identifier 1602 may be, for example, a unique alphanumeric code identifying a risk relationship between an enterprise (e.g., an insurer) and a party (e.g., an employee). The party identifier 1604 may be, for example, a unique alphanumeric code identifying an employee and may be based on, or associated with, the party identifier 1502 in the mental health database 1500. The equipment identifier 1604 may be, for example, a unique alphanumeric code identifying wearable device and may be based on, or associated with, the equipment identifier 1402 in the mental health equipment database 1400. The date 1606 may reflect when information was collected, when a treatment workflow was initiated, etc. The status 1610 might indicate “pending,” “active,” “closed,” etc. depending on the current situation of the employee.

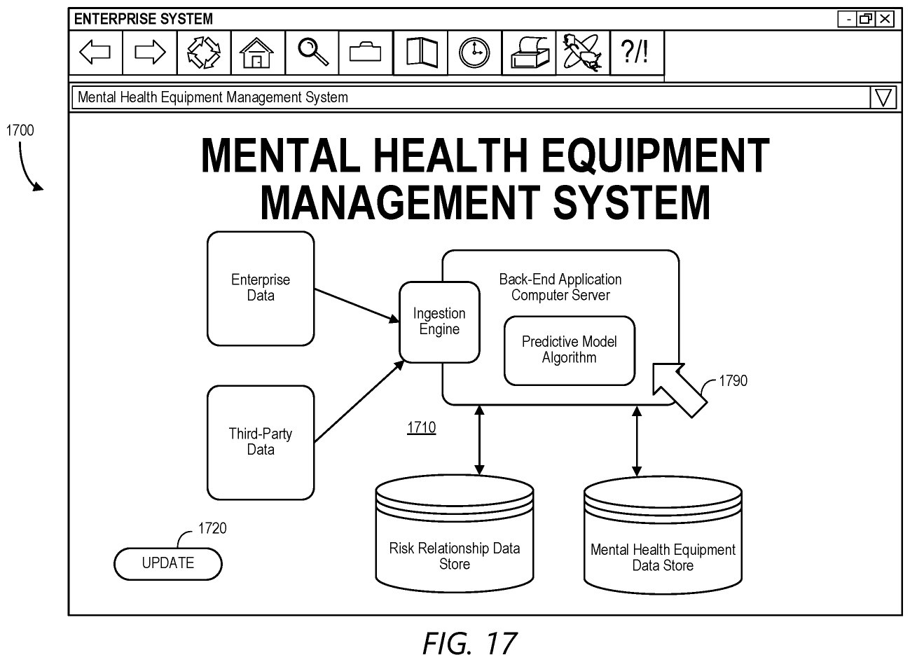

The operation of the mental health equipment management system and/or management tool may be controlled via a Graphical User Interface (“GUI”). For example, FIG. 17 is an equipment management operator or administrator display 1700 including graphical representations of elements of such a tool 1710 according to some embodiments. Selection of a portion or element of the display 1700 via a touchscreen or pointer 1790 might result in the presentation of additional information about that portion or element (e.g., a popup window presenting a data source or treatment workflow selection table) or let an operator or administrator enter or annotate additional information about resolution rules (e.g., based on his or her experience and expertise). An “Update” icon 1720 might initiate an equipment management process.

Thus, embodiments may provide an equipment management tool to let employees acknowledge challenges and seek support. The embodiments may provide real-time access to employees without requiring a prior referral from an employer or insurer.

The following illustrates various additional embodiments of the invention. These do not constitute a definition of all possible embodiments, and those skilled in the art will understand that the present invention is applicable to many other embodiments. Further, although the following embodiments are briefly described for clarity, those skilled in the art will understand how to make any changes, if necessary, to the above-described apparatus and methods to accommodate these and other embodiments and applications.

Although specific hardware and data configurations have been described herein, note that any number of other configurations may be provided in accordance with embodiments of the present invention (e.g., some of the information associated with the displays described herein might be implemented as a virtual or augmented reality display and/or the databases described herein may be combined or stored in external systems). Moreover, although embodiments have been described with respect to specific types of entities, embodiments may instead be associated with other types of businesses in additional to and/or instead of those described herein. Similarly, although certain types of insurance, treatment, and mental health parameters were described in connection some embodiments herein, other types of insurance products and/or parameters might be used instead.

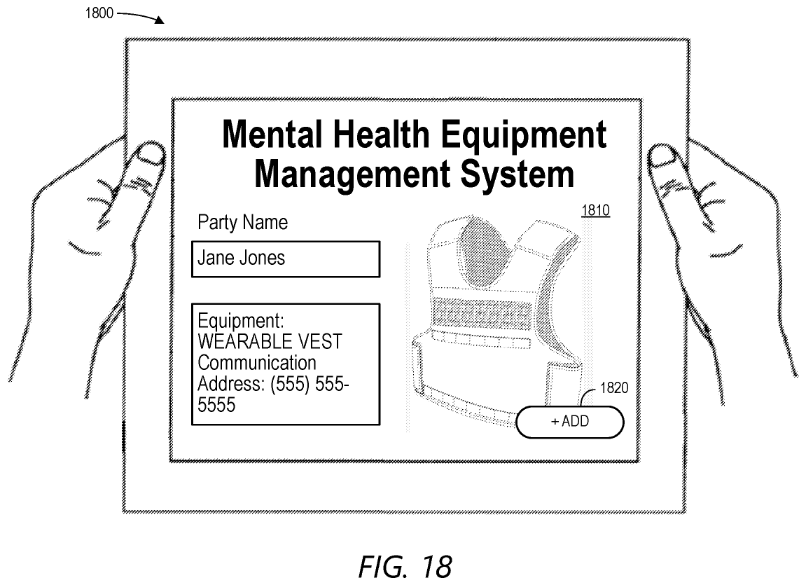

Note that the displays and devices illustrated herein are only provided as examples, and embodiments may be associated with any other types of user interfaces. For example, FIG. 18 illustrates a handheld tablet computer 1800 with an equipment management display 1810 according to some embodiments. The equipment management display 1810 shows one or more mental health devices that may be utilized by a user of the tablet computer 1800 (e.g., via a “+Add” icon 1820) to update an equipment management inventory.

According to some embodiments, one or more machine learning algorithms and/or predictive models may be used to perform automatic mental health decisions, select and execute treatment workflows, and/or identify potential mental health issues. Features of some embodiments associated with a predictive model will now be described by referring to FIG. 19. FIG. 19 is a partially functional block diagram that illustrates aspects of a computer system 1900 provided in accordance with some embodiments of the invention. For present purposes it will be assumed that the computer system 1900 is operated by an insurance company (not separately shown) for the purpose of supporting automated equipment management (e.g., to streamline the collection of and use of mental health information). According to some embodiments, the third-party data and/or risk relationship data may also be used to supplement and leverage the computer system 1900.

The computer system 1900 includes a data storage module 1902. In terms of its hardware the data storage module 1902 may be conventional, and may be composed, for example, by one or more magnetic hard disk drives. A function performed by the data storage module 1902 in the computer system 1900 is to receive, store and provide access to both historical data 1904 and current data 1906. As described in more detail below, the historical data 1904 is employed to train a predictive model to provide an output that indicates an identified performance metric and/or an algorithm to score or evaluate mental health decisions, and the current data 1906 is thereafter analyzed by the predictive model. Moreover, as time goes by, and results become known from processing current mental health decisions, at least some of the current decisions may be used to perform further training of the predictive model. Consequently, the predictive model may thereby adapt itself to changing conditions.

Either the historical data 1904 or the current data 1906 might include, according to some embodiments, determinate and indeterminate data. As used herein and in the appended claims, “determinate data” refers to verifiable facts such as an age of employee; an employee job type; an insurance policy date or other date; a time of day; a day of the week; a geographic location, address or ZIP code; and an insurance policy number.

As used herein, “indeterminate data” refers to data or other information that is not in a predetermined format and/or location in a data record or data form. Examples of indeterminate data include information from web sites, narrative speech or text, information in descriptive notes fields and signal characteristics in audible voice data files, etc.

The determinate data may come from one or more determinate data sources 1908 that are included in the computer system 1900 and are coupled to the data storage module 1902. The determinate data may include “hard” data like an entity name, date of incorporation, tax identifier number, insurance policy number, address, an underwriter decision, etc. One possible source of the determinate data may be the insurance company's policy database (not separately indicated).

The indeterminate data may originate from one or more indeterminate data sources 1910 and may be extracted from raw files or the like by one or more indeterminate data capture modules 1912. Both the indeterminate data source(s) 1910 and the indeterminate data capture module(s) 1912 may be included in the computer system 1900 and coupled directly or indirectly to the data storage module 1902. Examples of the indeterminate data source(s) 1910 may include data storage facilities for big data streams, document images, text files, and web pages. Examples of the indeterminate data capture module(s) 1912 may include one or more optical character readers, a speech recognition device (i.e., speech-to-text conversion), a computer or computers programmed to perform NLP, a computer or computers programmed to identify and extract information from images or video, a computer or computers programmed to detect key words in text files, and a computer or computers programmed to detect indeterminate data regarding an employee such as a questionnaire response, etc.

The computer system 1900 also may include a computer processor 1914. The computer processor 1914 may include one or more conventional microprocessors and may operate to execute programmed instructions to provide functionality as described herein. Among other functions, the computer processor 1914 may store and retrieve historical insurance data 1904 and current data 1906 in and from the data storage module 1902. Thus, the computer processor 1914 may be coupled to the data storage module 1902.

The computer system 1900 may further include a program memory 1916 that is coupled to the computer processor 1914. The program memory 1916 may include one or more fixed storage devices, such as one or more hard disk drives, and one or more volatile storage devices, such as RAM devices. The program memory 1916 may be at least partially integrated with the data storage module 1902. The program memory 1916 may store one or more application programs, an operating system, device drivers, etc., all of which may contain program instruction steps for execution by the computer processor 1914.

The computer system 1900 further includes a predictive model component 1918. In certain practical embodiments of the computer system 1900, the predictive model component 1918 may effectively be implemented via the computer processor 1914, one or more application programs stored in the program memory 1916, and computer stored as a result of training operations based on the historical data 1904 (and possibly also data received from a third party). In some embodiments, data arising from model training may be stored in the data storage module 1902, or in a separate computer store (not separately shown). A function of the predictive model component 1918 may be to determine appropriate performance metric scores, scoring algorithms, equipment management and treatment rules or decisions, etc. The predictive model component may be directly or indirectly coupled to the data storage module 1902.

The predictive model component 1918 may operate generally in accordance with conventional principles for predictive models, except, as noted herein, for at least some of the types of data to which the predictive model component is applied. Those who are skilled in the art are generally familiar with programming of predictive models. It is within the abilities of those who are skilled in the art, if guided by the teachings of this disclosure, to program a predictive model to operate as described herein.

Still further, the computer system 1900 includes a model training component 1920. The model training component 1920 may be coupled to the computer processor 1914 (directly or indirectly) and may have the function of training the predictive model component 1918 based on the historical data 1904 and/or information about entities. (As will be understood from previous discussion, the model training component 1920 may further train the predictive model component 1918 as further relevant data becomes available.) The model training component 1920 may be embodied at least in part by the computer processor 1914 and one or more application programs stored in the program memory 1916. Thus, the training of the predictive model component 1918 by the model training component 1920 may occur in accordance with program instructions stored in the program memory 1916 and executed by the computer processor 1914.

In addition, the computer system 1900 may include an output device 1922. The output device 1922 may be coupled to the computer processor 1914. A function of the output device 1922 may be to provide an output that is indicative of (as determined by the trained predictive model component 1918) particular mental health risk scores, equipment management rules or treatment decisions, etc. The output may be generated by the computer processor 1914 in accordance with program instructions stored in the program memory 1916 and executed by the computer processor 1914. More specifically, the output may be generated by the computer processor 1914 in response to applying the data for the current simulation to the trained predictive model component 1918. The output may, for example, be a numerical estimate, a likelihood within a predetermined range of numbers, a defined series of treatment steps, automatically generated alerts or suggestions, etc. In some embodiments, the output device may be implemented by a suitable program or program module executed by the computer processor 1914 in response to operation of the predictive model component 1918.

Still further, the computer system 1900 may include a mental health monitoring module 1924. The mental health monitoring module 1924 may be implemented in some embodiments by a software module executed by the computer processor 1914. The mental health monitoring module 1924 may have the function of rendering a portion of the display on the output device 1922. Thus, the mental health monitoring module 1924 may be coupled, at least functionally, to the output device 1922. In some embodiments, for example, the mental health monitoring module 1924 may direct communications with an enterprise by referring to an administrator 1928 via a mental health monitoring platform 1926, messages customized and/or generated by the predictive model component 1918 (e.g., suggesting treatment workflows, alerts or appropriate actions, etc.) and found to be associated with various parties or types of parties. In some embodiments, these results may be provided to the administrator 1928 who may also be tasked with determining whether or not performance may be improved.

The present invention has been described in terms of several embodiments solely for the purpose of illustration. Persons skilled in the art will recognize from this description that the invention is not limited to the embodiments described but may be practiced with modifications and alterations limited only by the spirit and scope of the appended claims.

What is claimed:

1. An equipment management system implemented via a back-end application computer server of an enterprise, comprising:
(a) a mental health equipment data store that contains electronic records associated with a plurality of mental health equipment identifiers, and, for each mental health equipment identifier, a communication address and associated equipment parameters;
(b) a risk relationship data store that contains electronic records associated with a plurality of parties having risk relationships with the enterprise, and, for each party, a party identifier, at least one mental health equipment identifier, and mental health data;
(c) the back-end application computer server, coupled to the mental health equipment data store and the risk relationship data store, including:
a computer processor,
a computer memory coupled to the computer processor and storing instructions that, when executed by the computer processor, cause the back-end application computer server to:
associate a selected equipment identifier in the mental health equipment data store with a selected party having a risk relationship with the enterprise,
update the risk relationship data store with the selected equipment identifier,
arrange to collect selected mental health data for the selected party in accordance with the communication address of the selected equipment identifier,
input the selected mental health data to a predictive model algorithm, and
automatically execute a treatment workflow for the selected party based on an output of the predictive model algorithm; and
(d) a communication port coupled to the back-end application computer server to facilitate an exchange of data with a remote device to support interactive user interface displays that provide information about the treatment workflow.

2. The system of claim 1, wherein the selected equipment identifier is based on at least one of: (i) a request from a party, (ii) a request from an entity associated with the party and an enterprise, and (iii) an analysis of mental health data.

3. The system of claim 1, wherein the selected equipment identifier is associated with at least one of: (i) a smartphone, (ii) a smartwatch, (iii) augmented reality glasses, (iv) a virtual reality display, (v) an application, and (vi) a web site.

4. The system of claim 1, wherein at least one equipment identifier is associated with wearable equipment.

5. The system of claim 4, wherein the wearable equipment is associated with at least one of: (i) a smartwatch, (ii) a bracelet, (iii) a ring, (iv) a vest, (v) a headband, (vi) a pillow, (vii) a mattress, (viii) a dongle, (ix) jewelry, (x) an identification credential, and (xi) a safety device.

6. The system of claim 1, wherein the treatment workflow is associated with at least one of: (i) an application, (ii) a return-to-work strategy, (iii) a referral, and (iv) an employee assistance program.

7. The system of claim 1, wherein the enterprise comprises an insurance company and each party comprises an employee of a business.

8. The system of claim 7, wherein the risk relationship is associated with at least one of: (i) an insurance policy, (ii) workers' compensation, (iii) long term disability, and (iv) short term disability.

9. The system of claim 1, wherein at least one communication address is associated with at least one of: (i) a telephone number, (ii) an email address, (iii) an Internet Protocol (“IP”) address, and (iv) Internet of Things (“IoT”) information.

10. An equipment management method implemented via a back-end application computer server of an enterprise, comprising:
associating, by a computer processor of a back-end application computer server, a selected equipment identifier in a mental health equipment data store with a selected party having a risk relationship with the enterprise, the mental health equipment data store containing electronic records associated with a plurality of mental health equipment identifiers, and, for each mental health equipment identifier, a communication address and associated equipment parameters;
updating a risk relationship data store with the selected equipment identifier, the risk relationship data store containing electronic records associated with a plurality of parties having risk relationships with the enterprise, and, for each party, a party identifier, at least one mental health equipment identifier, and mental health data;
arranging to collect selected mental health data for the selected party in accordance with the communication address of the selected equipment identifier;
inputting the selected mental health data to a predictive model algorithm; and
automatically executing a treatment workflow for the selected party based on an output of the predictive model algorithm.

11. The method of claim 10, wherein the selected equipment identifier is based on at least one of: (i) a request from a party, (ii) a request from an entity associated with the party and an enterprise, and (iii) an analysis of mental health data.

12. The method of claim 10, wherein the selected equipment identifier is associated with at least one of: (i) a smartphone, (ii) a smartwatch, (iii) augmented reality glasses, (iv) a virtual reality display, (v) an application, and (vi) a web site.

13. The method of claim 10, wherein at least one equipment identifier is associated with wearable equipment.

14. The method of claim 13, wherein the wearable equipment is associated with at least one of: (i) a smartwatch, (ii) a bracelet, (iii) a ring, (iv) a vest, (v) a headband, (vi) a pillow, (vii) a mattress, (viii) a dongle, (ix) jewelry, (x) an identification credential, and (xi) a safety device.

15. The method of claim 10, wherein the treatment workflow is associated with at least one of: (i) an application, (ii) a return-to-work strategy, (iii) a referral, and (iv) an employee assistance program.

16. A non-transitory, computer-readable medium storing instructions, that, when executed by a processor, cause the processor to perform an equipment management method implemented via a back-end application computer server of an enterprise, the method comprising:
associating, by a computer processor of a back-end application computer server, a selected equipment identifier in a mental health equipment data store with a selected party having a risk relationship with the enterprise, the mental health equipment data store containing electronic records associated with a plurality of mental health equipment identifiers, and, for each mental health equipment identifier, a communication address and associated equipment parameters;
updating a risk relationship data store with the selected equipment identifier, the risk relationship data store containing electronic records associated with a plurality of parties having risk relationships with the enterprise, and, for each party, a party identifier, at least one mental health equipment identifier, and mental health data;
arranging to collect selected mental health data for the selected party in accordance with the communication address of the selected equipment identifier;
inputting the selected mental health data to a predictive model algorithm; and
automatically executing a treatment workflow for the selected party based on an output of the predictive model algorithm.

17. The medium of claim 16, wherein the selected equipment identifier is associated with at least one of: (i) a smartphone, (ii) a smartwatch, (iii) augmented reality glasses, (iv) a virtual reality display, (v) an application, (vi) a web site, and (vii) wearable equipment.

18. The medium of claim 16, wherein the enterprise comprises an insurance company and each party comprises an employee of a business.

19. The medium of claim 18, wherein the risk relationship is associated with at least one of: (i) an insurance policy, (ii) workers' compensation, (iii) long term disability, and (iv) short term disability.

20. The medium of claim 16, wherein at least one communication address is associated with at least one of: (i) a telephone number, (ii) an email address, (iii) an Internet Protocol (“IP”) address, and (iv) Internet of Things (“IoT”) information.
